(19) United States
(12) Patent Application Publication
(10) Pub. No.: US 2021/0128778 A1
(43) Pub. Date: May 6, 2021

(54) SOLVENT DEPOSITION SYSTEM AND METHODS

(71) Applicants: BAXTER INTERNATIONAL INC., Deerfield, IL (US); BAXTER HEALTHCARE SA, Glattpark (Opfikon) (CH)

(72) Inventors: John Joseph Anthony BARRY, Northbrook, IL (US); Timothy Michael FULGHUM, Lakemoor, IL (US)

(21) Appl. No.: 16/491,774

(22) PCT Filed: Mar. 8, 2018
(86) PCT No.: PCT/US2018/021515

§ 371 Date: Sep. 6, 2019

Related U.S. Application Data
(60) Provisional application No. 62/469,163, filed on Mar. 9, 2017.

(51) Int. Cl. A61L 24/10; A61L 24/00; C08L 71/02

(52) U.S. Cl. CPC A61L 24/102; A61L 24/104; A61L 24/106; A61L 2420/02; A61L 24/0094; C08L 71/02; A61L 24/0036

(57) ABSTRACT

A hemostatic device comprising a biomaterial matrix and a polymeric material prepared by combining the polymeric material with a solvent, applying the combination to the biomaterial, removing the solvent, and retaining an effective layer of the polymeric material to the biomaterial to enhance the performance of the hemostatic device in the treatment of wounds.

### BACKGROUND

Hemostatic pads have been used for many years to improve wound healing or to stop bleeding. These pads may be made of biological materials such as collagen, gelatin, or oxidized cellulose, among other substances that act as biological glues or tissue sealants. For collagen pads, the mechanism of action in hemostasis is based on platelet aggregation and activation, the formation of thrombin on the surface of activated platelets and the formation of a hemostatic fibrin clot by the catalytic action of thrombin on fibrinogen. To improve the hemostatic action of hemostatic pads or sheets, factors of hemostasis may be included in the pads. For example, fibrinogen and/or factor XIII may be included. Thrombin, which enzymatically acts on fibrinogen to form fibrin, and on factor XIII to form the active factor XIIIa (which cross-links the fibrin to obtain a stable fibrin clot), may also be included in the pads, or as a separate component.

U.S. Pat. No. 8,703,170 describes hemostatic biomaterials coated or impregnated with an additional polymer to improve performance These biomaterials include flexible collagen pads with a three-dimensional structure which provides a matrix for additional strengthening of the clot when applied to a wound. The pad may be coated or impregnated with polyethylene glycol (PEG), NHS-PEG, or another PEG derivative. The pad works similarly to those known in the state of the art or available on the market, such as Hemopatch Healing Hemostat.

WO2004028404 describes a tissue sealant composed of a synthetic collagen or gelatin and an electrophilic cross-linking agent which are provided in a dry state. Upon wetting of this composition at an appropriate pH, a reaction between the two components takes place and a gel with sealing properties is formed. Such a sealant works essentially analogously to two component sealants (composed of a reagent with multiple electrophilic groups and a reagent with multiple nucleophilic groups) which are known in the state of the art or which are available on the market, e.g. Coseal™. In an embodiment, the two components of the sealant (the electrophilic cross-linker and the synthetic collagen/gelatin) are coated onto a biomaterial.

WO 97/37694 discloses a hemostatic sponge based on collagen and an activator or proactivator of blood coagulation homogeneously distributed therein. This sponge is provided in a dry form, which could be air-dried or lyophilized However, it still contains a water content of at least 2%.

In U.S. Pat. No. 4,600,574 a tissue adhesive based on collagen combined with fibrinogen and factor XIII is described. This material is provided in the lyophilized form, ready for use. The fibrinogen and factor XIII are combined with the collagen by impregnating the collagenous flat material with a solution comprising fibrinogen and factor XIII, and lyophilizing said material.

U.S. Pat. No. 5,614,587 discusses bioadhesive compositions comprising cross-linked collagen using a multifunctionally activated synthetic hydrophilic polymer, as well as methods of using such compositions to effect adhesion between a first surface and a second surface, wherein at least one of the first and second surfaces can be a native tissue surface.

Collagen-containing compositions which have been mechanically disrupted to alter their physical properties are described in U.S. Pat. Nos. 5,428,024, 5,352,715, and 5,204,382. These patents generally relate to fibrillar and insoluble collagens. An injectable collagen composition is described in U.S. Pat. No. 4,803,075. An injectable bone/cartilage composition is described in U.S. Pat. No. 5,516,532. A collagen-based delivery matrix comprising dry particles in the size range from 5 μm to 850 μm which may be suspended in water and which has a particular surface charge density is described in WO 96/39159. A collagen preparation having a particle size from 1 μm to 50 μm useful as an aerosol spray to form a wound dressing is described in U.S. Pat. No. 5,196,185. Other patents describing collagen compositions include U.S. Pat. Nos. 5,672,336 and 5,356,614.

### SUMMARY

The example hemostatic devices including sponges, patches, pads, sealants and methods of manufacturing such devices disclosed herein are especially suitable for stopping bleeding and for wound healing. The hemostatic devices and surgical sealants are also useful for procedures in which control of bleeding or leakage of other body fluids or air by conventional surgical techniques is either ineffective or impractical.

It has been found that previous pads of fibrous biomaterials, in particular collagen pads, for wound healing failed to induce hemostasis at conditions with impaired hemostasis (e.g. after heparinization). The device according to the present invention improves hemostasis. Furthermore, the device according to the present invention shows a strong adherence to the tissue when applied to a wound. The device of the present invention further shows improved swelling behavior, i.e. low swelling, after application to a wound.

A further aspect relates to a method of manufacturing such sponges or devices.

The invention includes hemostatic devices manufactured in accordance with the present invention

In one aspect, embodiments of the present invention encompass a hemostatic pad. Exemplary pads may include a matrix of a biomaterial and one hydrophilic polymeric component having reactive groups. The biomaterial and polymeric component can be associated with each other so that the reactivity of the polymeric component is retained. The biomaterial and polymeric component can be associated so that the polymeric component is coated onto a surface of said matrix of a biomaterial, or so that the matrix is impregnated with the polymeric material, or both. The polymeric material is combined with a solvent and may be sprayed or printed on the surface of biomaterial. The solvent is then removed, leaving the biomaterial coated with the polymeric material. The biomaterial can include collagen, gelatin, fibrin, a polysaccharide, e.g. chitosan, a synthetic biodegradable biomaterial, e.g. polylactic acid or polyglycolic acid, and derivatives thereof. The hydrophilic polymer can be a polyalkylene oxide polymer, esp. preferred a PEG comprising polymer, e.g. a multi-electrophilic polyalkylene oxide polymer, e.g. a multi-electrophilic PEG, such as pentaerythritolpoly(ethyleneglycol)ether tetrasuccinimidyl glutarate. In some cases, the biomaterial can be collagen and the polymeric component can be pentaerythritolpoly(ethyleneglycol)ether tetrasuccinimidyl glutarate. The polymeric form can be coated onto the collagen. In some cases, the biomaterial is collagen and the polymeric component is pentaerythritolpoly(ethyleneglycol)ether tetrasuccinimidyl glutarate, and the polymeric form is impregnated into the collagen.

In an embodiment, the biomaterial is coated with PEG or a derivative thereof. The PEG is combined with a non-reactive solvent with a low boiling point to dissolve the PEG and then the solvent and PEG combination is sprayed or printed on the surface of the matrix in a uniform or patterned coating. The solvent is then removed, leaving behind the PEG layer. In an embodiment, the solvent is removed through low pressure or low temperature evaporation.

In an embodiment, no thermal processing is needed in the manufacture of the device.

Additional features and advantages of the disclosed hemostatic device and methods of manufacture are described in, and will be apparent from, the following Detailed Description.

Those skilled in the art will readily understand that all embodiments disclosed in the following are examples of specific embodiments, but are not necessarily limiting the general inventive concept. Furthermore, all exemplary embodiments can be read on all inventive aspects and embodiments in any combination, if not mutually exclusive. All equivalents or obvious alterations or modifications as recognized by those skilled in the art are included by the present invention.

### DETAILED DESCRIPTION OF THE INVENTION

The following abbreviations are used:
COH102 Pentaerythritolpoly(ethyleneglycol)ether tetrasuccinimidyl glutarate
COH206 Pentaerythritolpoly(ethyleneglycol)ether tetra-thiol
EtOH ethanol
PEG polyethylene glycol
PET polyethylene terephthalate.

The object of the invention is a hemostatic device comprising a biomaterial and a polymeric material or component applied to the biomaterial. The biomaterial may be a fibrous biomaterial. The device is prepared by combining the polymeric material with a solvent, applying the polymeric material and solvent to the biomaterial and removing the solvent, leaving a coating of the polymeric material on the biomaterial.

Preferably the biomaterial is collagen, a protein, a biopolymer, or a polysaccharide. Preferably, the biomaterial is a biomaterial selected from the group consisting of collagen, gelatin, fibrin, oxidized cellulose, a polysaccharide, e.g. chitosan, and a derivative thereof, more preferred collagen and chitosan, especially preferably collagen.

The device is a porous network of a biomaterial able to absorb body fluids when applied to the site of an injury. Furthermore, the device is usually flexible and suitable to be applied on diverse tissues and locations with various shapes.

The collagen used for the present invention can be from any collagen suitable to form a gel, including a material from liquid, pasty, fibrous or powdery collageneous materials that can be processed to a porous or fibrous matrix. The preparation of a collagen gel for the production of a sponge is e.g. described in the EP 0891193 (incorporated herein by reference) and may include acidification until gel formation occurs and subsequent pH neutralisation. To improve gel forming capabilities or solubility the collagen may be (partially) hydrolyzed or modified, as long as the property to form a stable sponge when dried is not diminished.

The collagen sponge according to the present invention preferably has a lower density as compared to the density of a collagen film. Preferably the density is between about 5 to about 100 mg per cm³, whereas densities of films are higher than about 650 mg per cm³. An especially preferred collagen sponge according to the present invention is the one marketed under the name Matristypt®.

The collagen or gelatin of the sponge matrix is preferably of animal origin, preferably bovine or equine. However, human collagen can be used in case of a hypersensitivity of the patient towards xenogenic proteins. The further components of the sponge are preferably of human origin, which makes the sponge suitable especially for the application to a human.

In an embodiment the matrix material of the fibrous biocompatible polymer which forms the porous network of the sponge constitutes of between 1-50%, 1-10%, preferably about 3% of the dried porous sponge (w/w-%).

In an embodiment, the fibrous biomaterial has particles of a fluid absorbing particulate material adhered to the matrix. The fluid absorbing particulate material may comprise a hydrophilic polymeric component that may be a cross-linked polymer.

In a preferred embodiment the polymeric component is a single hydrophilic polymer component that is a crosslinker, in the following called “the material”. The hydrophilic polymeric component may be a crosslinker, especially a polyalkylene oxide polymer. A PEG comprising polymer is especially preferred. The reactive groups of said material are preferably electrophilic groups.

Preferred electrophilic groups of the hydrophilic polymeric crosslinker according to the present invention are groups reactive to the amino-, carboxy-, thiol- and hydroxy- groups of proteins, or mixtures thereof.

Preferred amino group-specific reactive groups are NHS-ester groups, NBS-ester groups, imidoester groups, aldehyde-groups, carboxy-groups in the presence of carbodiimdes, isocyanates, or THPP (beta-[Tris(hydroxymethyl)phosphino]propionic acid), especially preferred is pentaerythritolpoly(ethyleneglycol)ether tetrasuccinimidyl glutarate (═Pentaerythritol tetrakis[1-1′-oxo-5′-succinimidylpentanoate-2-poly-oxoethyleneglycol ether (═an NHS-PEG)), particularly an NHS-PEG with MW 10,000.

Preferred carboxy-group specific reactive groups are amino-groups in the presence of carbodiimides. Preferred thiol group-specific reactive groups are maleiimides or haloacetyls. A preferred hydroxy group-specific reactive group is the isocyanate group.

The reactive groups on the hydrophilic cross-linker may be identical (homo-functional) or different (hetero-functional). The hydrophilic polymeric component can have two reactive groups (homo-bifunctional or heterobifunctional) or more (homo/hetero-trifunctional or more).

In special embodiments the material is a synthetic polymer, preferably comprising PEG. The polymer can be a derivative of PEG comprising active side groups suitable for cross-linking and adherence to a tissue. By the reactive groups the hydrophilic polymer has the ability to cross-link blood proteins and also tissue surface proteins. Cross-linking to the biomaterial is also possible.

The multi-electrophilic polyalkylene oxide may include two or more succinimidyl groups. The multi-electrophilic polyalkylene oxide may include two or more maleimidyl groups. Preferably, the multi-electrophilic polyalkylene oxide is a polyethylene glycol or a derivative thereof. In a preferred embodiment, the polymeric component is pentaerythritolpoly(ethyleneglycol)ether tetrasuccinimidyl glutarate (═COH102, also pentaerythritol tetrakis[1-1′-oxo-5′-succinimidylpentanoate-2-poly-oxoethyleneglycol]ether).

In an embodiment the polymeric material, in the following called “the material”, is a mixture of two pre-polymers comprising a first cross-linkable component and a second cross-linkable component that cross-links with the first cross-linkable component under reaction enabling conditions or a formed polymer in association with said sponge.

More preferably said first and/or second cross-linkable component comprise a derivative of polyethylene glycol (PEG), e.g. a derivative which is able to react under given conditions. Preferably one of the cross-linkable components is capable of covalently reacting with tissue.

Such materials suitable for a sponge for use as a hemostat are e.g. disclosed in W02008/016983 (incorporated herein by reference in its entirety) and commercially available under the trademark CoSeal®. Preferred materials mediate adjunctive hemostasis by themselves, and can be suitable to mechanically seal areas of leakage. Such materials are for example bioresorbable polymers, in particular polymers that cross-link and solidify upon exposure to body fluids. In further embodiments the material is resorbable and/or biocompatible and can be degraded by a subject, in particular a human subject, in less than 6 months, less than 3 months, less than 1 month or less than 2 weeks.

A suitable polymeric material may comprise a first cross-linkable component, a second cross-linkable component that cross-links with the first cross-linkable component under reaction enabling conditions, wherein the first and second cross-linkable component cross-link to form a layer.

The first cross-linkable component can include multiple nucleophilic groups and the second cross-linkable component can include multiple electrophilic groups. Upon contact with a biological fluid, or in other reaction enabling conditions, the cross-linkable first and second components cross-link to form a porous matrix having interstices.

In some aspects, the first cross-linkable component of the material includes a multi-nucleophilic polyalkylene oxide having m nucleophilic groups, and the second cross-linkable component includes a multi-electrophilic polyalkylene oxide. The multi-nucleophilic polyalkylene oxide can include two or more nucleophilic groups, for example NH₂, —SH, —OH, —H, —PH₂, and/or —CO—NH—NH₂. In some cases, the multi-nucleophilic polyalkylene oxide includes two or more primary amino groups. In some cases, the multi-nucleophilic polyalkylene oxide includes two or more thiol groups. The multi-nucleophilic polyalkylene oxide can be polyethylene glycol or a derivative thereof. In some cases, the polyethylene glycol includes two or more nucleophilic groups, which may include a primary amino group and/or a thiol group. The multi-electrophilic polyalkylene oxide can include two or more electrophilic groups such as succinimidyl esters (—CO₂N(COCH₂)₂), carboxylic acids (—CO₂H), aldehydes (—CHO), epoxides (—CHOCH₂), isocyanates (—N═C═O), vinyl sulfones (—SO₂CH═CH₂), maleimides (—N(COCH)₂), and/or pyridyl disulfides (—S—S—(C₅H₄N)). The multi-electrophilic polyalkylene oxide may include two or more succinimidyl groups. The multi-electrophilic polyalkylene oxide may include two or more maleimidyl groups. In some cases, the multi-electrophilic polyalkylene oxide can be a polyethylene glycol or a derivative thereof.

In certain embodiments the first and/or second cross-linkable component is/are synthetic polymers, preferably comprising PEG. The polymer can be a derivative of PEG comprising active side groups suitable for cross-linking and adherence to a tissue. Preferably, the adhesive comprises succinimidyl, maleimidyl and/or thiol groups. In a two polymer set-up, one polymer may have succinyl or maleimidyl groups and a second polymer may have thiol or amino groups which can attach to the groups of the first polymer. These or additional groups of the adhesive may facilitate the adherence to a tissue. The adhesive layer may comprise one or more cross-linked components.

The polymeric material, such as modified PEG material as mentioned before, is present in a range of 0.5 to 50 mg/cm² of the biomaterial, preferably 2 to 20 mg/cm² of the biomaterial, e.g. collagen.

The sponge as a whole is biodegradable, being suitable for biological decomposition in vivo, or bioresorbable, i.e. able to be resorbed in vivo. Full resorption means that no significant extracellular fragments remain. A biodegradable material differs from a non-biodegradable material in that a biodegradable material can be biologically decomposed into units which may either be removed from the biological system and/or chemically incorporated into the biological system. In a preferred embodiment the particular material, the matrix material or sponge as a whole can be degraded by a subject, in particular a human subject, in less than 6 month, less than 3 month, less than 1 month, less than 2 weeks.

In an embodiment the sponge has the material enhancing the adherence of said sponge to the applied tissue in the form of a continuous or discontinuous layer on at least one surface of said sponge.

The device of the present invention preferably has an overall thickness of less than 2.5 mm, more preferred about 1 mm to about 2.5 mm.

The device of the present invention is preferably used in minimal invasive surgery, e.g. for laparoscopic application.

The device may be dried and after drying, the sponge may have a water content of at least 0.5 (as a w/w percentage). In certain embodiments the sponge can be freeze-dried or air-dried.

The device may further comprise an activator or proactivator of blood coagulation, including fibrinogen, thrombin or a thrombin precursor, as e.g. disclosed in U.S. Pat. No. 5,714,370 (incorporated herein by reference). Thrombin or the precursor of thrombin is understood as a protein that has thrombin activity and that induces thrombin activity when it is contacted with blood or after application to the patient, respectively. Its activity is expressed as thrombin activity (NIH-Unit) or thrombin equivalent activity developing the corresponding NIH-Unit. The activity in the sponge can be 100-10,000, preferably 500-5,000. In the following thrombin activity is understood to comprise both, the activity of thrombin or any equivalent activity. A protein with thrombin activity might be selected from the group consisting of alpha-thrombin, meizothrombin, a thrombin derivative or a recombinant thrombin. A suitable precursor is possibly selected from the group consisting of: prothrombin, factor Xa optionally together with phospholipids, factor IXa, activated prothrombin complex, FEIBA, any activator or a proactivator of the intrinsic or extrinsic coagulation, or mixtures thereof.

The hemostatic device according to the invention might be used together with further physiologic substances. For example, the device preferably further comprises pharmacologically active substances, among them antifibrinolytics, such as a plasminogenactivator-inhibitor or a plasmin inhibitor or an inactivator of fibrinolytics. A preferred antifibrinolytic is selected from the group consisting of aprotinin or an aprotinin derivative, alpha2-macroglobulin, an inhibitor or inactivator of protein C or activated protein C, a substrate mimic binding to plasmin that acts competitively with natural substrates, and an antibody inhibiting fibrinolytic activity.

As a further pharmacologically active substance an antibiotic, such as an antibacterial or antimycotic might be used together with the device according to the invention, preferably as a component homogeneously distributed in the device. Further bioactive substances such as growth factors and/or pain killers may be also present in the inventive device. Such a device might be useful in e.g. wound healing. Further combinations are preferred with specific enzymes or enzyme inhibitors, which may regulate, i.e. accelerate or inhibit, the resorption of the device. Among those are collagenase, its enhancers or inhibitors. Also, a suitable preservative may be used together with the device or may be contained in the device.

Although an embodiment relates to the use of the hemostatic device which contains the activator or proactivator of blood coagulation as the only active component, further substances that influence the velocity of blood coagulation, hemostasis and quality of the sealing, such as tensile strength, inner (adhesive) strength and durability might be comprised.

Procoagulants that enhance or improve the intrinsic or extrinsic coagulation, such as factors or cofactors of blood coagulation, factor XIII, tissue factor, prothrombin complex, activated prothrombin complex, or parts of the complexes, a prothrombinase complex, phospholipids and calcium ions, might be used. In case of a surgical procedure where a precise sealing is needed, it might be preferable to prolong the working period after the hemostatic device is applied to the patient and before clotting is affected. The prolongation of the clotting reaction will be ensured, if the device according to the invention further comprises inhibitors of blood coagulation in appropriate amounts. Inhibitors, such as antithrombin III optionally together with heparin, or any other serine protease inhibitor, are preferred.

It is also preferred to have such additives, in particular the thrombin or a precursor of thrombin evenly distributed in the material in order to prevent local instability or hypercoagulability of the material. Even with a certain water content the thrombin activity is surprisingly stable, probably because of the intimate contact of thrombin and collagen in the homogeneous mixture. Nevertheless, thrombin stabilizers preferably selected from the group consisting of a polyol, a polysaccharide, a polyalkylene glycol, amino acids or mixtures thereof might be used according to the invention. The exemplary use of sorbitol, glycerol, polyethylene glycol, polypropylene glycol, mono- or disaccharides such as glucose or saccharose or any sugar or sulfonated amino acid capable of stabilizing thrombin activity is preferred. A pH of approximately 6.0 is preferred.

In another embodiment a biocompatible, resorbable hydrogel capable of absorbing liquid is contained within the device of the present invention.

The present invention also provides a wound coverage comprising a device according to the invention. The device and all additional layers can be provided in a ready to use wound coverage in suitable dimensions. The device and/or the coverage can be a sponge pad or a sheet, preferably having a thickness of at least 3 mm or at least 5 mm and/or up to 20 mm, depending on the indication. When the relatively thick flexible sponge is applied to a wound it is important that blood and fibrinogen can be absorbed throughout the sponge before fibrin is formed that might act as a barrier for the absorption of further wound secret.

Another aspect of the invention relates to a method of manufacturing a hemostatic porous device comprising
a) providing a device comprising a matrix of a biomaterial,
b) providing a polymeric material in the form of a suspension, a solution or powder in combination with a non-reactive solvent;
c) contacting a) and b) so that the material of b) is present on at least one surface of said device,
d) removing the solvent, leaving behind the polymeric material on the at least one surface of the device; and optionally
e) drying the device obtained in step d).

Drying may include freeze drying or air drying and comprises removing volatile components of the fluid.

The solvent and polymeric material combination may be contacted with the biomaterial by rolling, spraying, printing, painting, or via film adherence. In an embodiment, either a gas assisted sprayer or gasless sprayer is used.

If the polymeric material is applied in powdered form, a final crystallization step may be used. If the polymeric material is applied in solubilized form, the polymeric material may be directly sprayed on the biomaterial.

The polymeric material may cover the biomaterial matrix in the range of 2-20 mg/cm².

In an embodiment, aerosolized reactive PEG is sprayed on collagen or another biomaterial in order such a manner as to retain the reactivity of the PEG. It is preferable for the spraying to occur over a short period of time.

The solvent may a non-reactive solvent. Preferably, the solvent may be a biocompatible solvent. In an embodiment, the solvent is an organic aprotic solvent such as acetone, toluene, dicholoromethane, chloroform, ethyl acetate, dimethyl sulfoxide, etc. The solvent may also be an alcohol such as ethanol, methanol, or isopropanol. If a protic solvent is used, in an embodiment, the protic solvent is acidified. A combination of solvents may be used.

In a preferred embodiment, the solvent may be acetone, ethyl acetate, dimethyformamide or dimethyl sulfoxide.

In an embodiment, the solvent is removable by using high flow gas, light, heat, or vacuum. In an embodiment, the solvent is removable in a vacuum chamber.

In an embodiment, the solvent may be flashed off, leaving the polymeric material behind.

Certain steps of the method of manufacturing the hemostatic device may be performed in an inert atmosphere. In an embodiment, the device is sterilized and processed in an inert atmosphere. In an embodiment the device is package in an inert atmosphere. In another embodiment, the entire manufacturing process takes place in an inert atmosphere.

The present invention allows for uniform or patterned particle dissolution of the polymeric material over the biomaterial matrix in a smooth application. The method of applying the polymeric material leads to a coating of an appropriate thickness to enhance adherence of the hemostatic device to a wound surface. It also reduces waste as compared to standard methods of manufacture for hemostatic devices. Further, the present invention is safer than standard methods, as there is no need for thermal processing and removes the risk of thermal stressors on the device during manufacture.

The method further allows for effective penetration of the polymeric material into the biomaterial (e.g., PEG into collagen) to improve adherence of the biomaterial to tissue. Uniform coatings may be achieved more effectively than through other methods. The method also allows for enhanced infiltration of the polymeric material into the biomaterial matrix as compared to standard methods of manufacture while minimizing impurities. The resultant device is substantially free of degradants resulting from thermal stress.

In a further aspect the present invention provides a hemostatic device obtainable by the method according to the invention described above. All embodiments mentioned above for a hemostatic device can also be read to this obtainable device.

The present invention also provides a method of treating an injury comprising administering a hemostatic device comprising a matrix of a biomaterial and a polymeric material obtainable by the method according to the invention described above. The injury may comprise a wound, a hemorrhage, damaged tissue and/or bleeding tissue.

The present invention is further exemplified by the following examples without being limited thereto.

### EXAMPLES

### Example 1: Collagen Sponges Treated With Acidic Solution of Two Reactive PEGs

Aqueous, acidic solutions (pH 3.0, HCl) of COH102 and COH206 with PEG-concentrations (COH102 and COH206 1:1) of 10 mg/cm³, 35 mg/cm³, 70 mg/cm³ and 100 mg/cm³ are prepared in combination with a suitable solvent such as acetone, ethyl acetate, dimethyformamide or dimethyl sulfoxide. The solutions with the solvent are sprayed on commercial available bovine collagen sponges (Matristypt®), 9×7 cm.

The solvent is flashed off, leaving a coating of reactive PEGs in an organized pattern on the collagen.

After lyophilization the dried sponges may be packed together with desiccants in water vapor impermeable pouches and may be further gamma-sterilized, e.g. with 25 kGray.

The entire process occurs in an inert environment

### Example 2: Collagen Sponges Treated With EtOH-Solution of Two Reactive PEGs

COH102 and COH206 are dissolved in completely dried EtOH. PEG-concentrations (COH102 and COH206 1:1) of 10 mg/cm³, 35 mg/cm³, 70 mg/cm³ and 100 mg/cm³ are prepared are prepared in combination with a suitable solvent. The solutions with the solvent are sprayed on commercial available bovine collagen sponges (Matristypt®), 9×7 cm.

The collagen materials are placed in a vacuum chamber, removing the solvent.

Dried sponges may be packed together with desiccants in water vapor impermeable pouches and may be gamma-sterilized, e.g. with 25 kGray.

### Example 3: Preparation of Collagen-/Reactive PEG Constructs

22 ml of aqueous, acidic solutions (pH 3.0, HCl) containing various concentrations (2.15 mg/cm³, 4.3 mg/cm³ and 7.2 mg/cm³ of bovine corium collagen and PEG (COH102 and COH206 1:1)-concentrations of 7.2 mg/cm³, 14.3 mg/cm³, 28.6 mg/cm³ and 57.3 mg/cm³ are prepared in combination with a suitable solvent.

The solvent is flashed off, leaving a coating of reactive PEGs in an organized pattern on the collagen.

After lyophilization the dried sponges may be packed together with desiccants in water vapor impermeable pouches and may be further gamma-sterilized, e.g. with 25 kGray.

### Example 4: Preparation of Two Layer Collagen-/Reactive PEG Constructs

11 ml and 22 ml of acidic collagen-/PEG-solutions (pH 3.0, HCl) as described in example 3 are filled into PET-trays and immediately frozen at −20° C. On the top of the ice phase 11 ml or 22 ml of a 1% bovine corium collagen solution, pH 3.0 (HCl) are applied and the constructs obtained are freeze-dried.

The dried sponges may be packed together with desiccants in water vapor impermeable pouches and may be gamma-sterilized, e.g. with 25 kGray.

Example 5: Homogeneous Coating of Collagen Sponges With Reactive PEGs

A 1:1 powder mixture of COH102 and COH206 is homogeneously distributed onto one surface of a commercially available collagen sponge or on a sponge prepared after one of the methods as described in example 1, 2, 3 and 4. PEG-amounts of 2 mg/cm², 7 mg/cm², 10 mg/cm², 14 mg/cm² and 20 mg/cm² are used for the coating. The PEG-powder mixture is combined with a solvent and sprayed, painted, or printed onto the collagen. The solvent is flashed off, leaving the collagen uniformly coated with PEG. The sponges are then lyophilized.

The dried sponges may be packed together with desiccants in water vapor impermeable pouches and may be gamma-sterilized, e.g. with 25 kGray.

### Example 6: Discontinuous Coating of Collagen Sponges With Reactive PEGs

Pads are prepared as described in example 5 with the exception that before spraying, printing or painting the PEG and solvent solution on the collagen a grid is placed onto the surface of the collagen sponge, so that the surface of the pad is partially shielded and partially not covered by the PEG powder. Grid matrices with a mesh size of 5 mm and 1O mm are used and removed after distribution. Removal of the solvent, fixation of the powder, packaging and sterilization are those as described in example 5.

These prototypes allow a better penetration of the blood into the collagen pad, where coagulation occurs due to the procoagulant activity of collagen. The reactive PEGs assure the adhesion of the pad to the wound surface.

### Example 7: Preparation of Constructs of Collagen With Cross-Linked PEG

a) Onto a bovine collagen sponge the reactive PEGs COH102 and COH206 (1:1) in combination with a solvent are sprayed with a commercial available spray applicator composed of a double syringe and a gas driven spray head (Duplospray, Baxter). One syringe contains COH102 and COH206 at pH 3.0 in combination with the solvent and the second syringe buffer, pH 9.4. The polymerization of the two PEG-components occurs on the surface of collagen immediately after deposition. The solvent is then removed. The sponge may be dried in a vacuum chamber.

b) A collagen sponge is treated with an acidic PEG-solution as described in example 1. In order to start the cross-linking between the two PEG-components and the collagen matrix, the wet sponge is treated with a basic buffer system and may be lyophilized afterwards.

### Example 8: Continuous Coating of a Chitosan-/Gelatin Sponge With Reactive PEG's

A 1:1 powder mixture of COH102 and COH206 and a solvent is homogeneously distributed onto one surface of a commercially available chitosan-/gelatin (Chitoskin®, Beese Medical) sponge. A PEG-amount of 14 mg/cm2 is used for the coating. The PEG-powder mixture is fixed on the surface of the sponge and the solvent is removed. The sponge is then lyophilized.

The dried sponges may be packed together with desiccants in water vapor impermeable pouches and may be gamma-sterilized, e.g. with 25 kGray.

### Example 9: Coating of an Oxidized Cellulose Fabric With Reactive PEG's

A 1:1 powder mixture of COH102 and COH206 in combination with a solvent is distributed via spraying, printing or painting onto one surface of a commercially available oxidized cellulose fabric (Traumstem®, Bioster). A PEG-amount of 14 mg/cm² is used for the coating. The solvent is then flashed off and the sponges are lyophilized.

The dried sponges may be packed together with desiccants in water vapor impermeable pouches and may be gamma-sterilized, e.g. with 25 kGray.

### Example 10: Preclinical Applications

A sponge as prepared according to the examples is tested in heparinized pigs (1.5-fold ACT) in a liver abrasion model. With a rotating grinding machine a circular bleeding wound with a diameter of 1.8 cm is created on the surface of a liver lobe. A 3×3 cm sponge is applied and moderately pressed against the wound for 2 minutes with a piece of gauze soaked with saline buffer. After removal of the gauze a good hemostatic performance is achieved.

### Example 11: Cross-Sectioning and Purity

A sponge as prepared according to the examples is cross-sectioned The cross section demonstrates deeper penetration depth of the polymeric material into the biomaterial as compared to other methods of fixing the polymeric material (e.g. as compared to thermal processing). A particulate matter and heavy metals test is run that demonstrates that the sponges as prepared according to the examples have few impurities.

The invention is claimed as follows:

1: A hemostatic sponge comprising a matrix of a biomaterial and a polymeric material, wherein the polymeric material uniformly coats at least one surface of the biomaterial and wherein the device is substantially free of degradants caused by thermal stress.

2: The sponge according to claim 1, wherein the biomaterial is selected from the group consisting of collagen, gelatin, fibrin, oxidized cellulose, a polysaccharide, chitosan, and derivatives thereof.

3: The sponge according to claim 2, wherein the biomaterial is collagen.

4: The sponge according to any one of claims 1 to 3, wherein the polymeric material is reactive polyethylene glycol.

5: The sponge according to any one of claims 1 to 3, wherein the polymeric material is a single hydrophilic polymeric crosslinker.

6: A method of manufacturing a hemostatic device comprising
providing a porous sponge of a matrix of a biomaterial;
providing a polymeric material in the form of a suspension, solution, or powder;
combining the polymeric material with a solvent;
contacting the combination of the polymeric material and the solvent with at least one surface of the biomaterial matrix;
removing the solvent; and
retaining a coating of the polymeric material on the biomaterial matrix.

7: The method of claim 6, wherein the polymeric material is reactive polyethylene glycol.

8: The method of claim 6 or 7, wherein the solvent is an organic aprotic solvent.

9: The method of claim 6 or 7, wherein the solvent is an acidified protic solvent.

10: The method of any one of claims 6 to 9, wherein the combination of the polymeric material and the solvent are contacted with the biomaterial matrix by a method selected from the group consisting of printing, spraying, painting, film adhesion, and combinations thereof.

11: The method of claim 10, wherein the combination of the polymeric material and the solvent is sprayed on the biomaterial matrix with a gas-assisted sprayer.

12: The method of any one of claims 6 to 11 wherein the coating of the polymeric material retained on the biomaterial matrix has thickness of 2-20 mg/cm².

13: A hemostatic device comprising a biomaterial matrix and a polymeric material, wherein the polymeric material is stably associated with biomaterial matrix through the steps of
combining the polymeric material with a solvent;
applying the polymeric material and the solvent to the biomaterial matrix; and
subsequently removing the solvent.

14: The device of claim 13, wherein the polymeric material is reactive polyethylene glycol.

15: The device of claim 13 or 14, wherein the solvent is an organic aprotic solvent.

16: The device of claim 13 or 14, wherein the solvent is an acidified protic solvent.

17: The device of any one of claims 13 to 16, wherein the polymeric material and the solvent are applied by a method selected from the group consisting of printing, spraying, painting, film adhesion, and combinations thereof.

18: The device of claim 17, wherein the polymeric material and the solvent is sprayed on the biomaterial matrix with a gas-assisted sprayer.

19: The device of claim any one of claims 13 to 18, wherein the polymeric material has thickness of 2-20 mg/cm².

20: The device of claim any one of claims 13 to 19, wherein the device is substantially free of impurities.
